(19) United States
(12) Patent Application Publication
(10) Pub. No.: US 2012/0283524 A1
(43) Pub. Date: Nov. 8, 2012

(54) PEDIATRIC MONITOR SENSOR STEADY GAME

(75) Inventors: Abraham M. Kiani, San Juan Capistrano, CA (US); Massi Joe E. Kiani, Laguna Niguel, CA (US); Marcelo M. Lamego, Coto De Caza, CA (US)

(73) Assignee: CERCACOR LABORATORIES, INC., Irvine, CA (US)

(21) Appl. No.: 13/450,380

(22) Filed: Apr. 18, 2012

Related U.S. Application Data
(60) Provisional application No. 61/476,694, filed on Apr. 18, 2011.

(51) Int. Cl. A61B 5/11; A61B 6/00

(52) U.S. Cl. 600/301

(57) ABSTRACT

A monitoring device for measuring one or more physiological parameters of a medical patient can include a finger clip sensor connected to a monitor. An accelerometer in the finger clip sensor detects patient motion or movement that may interfere with monitoring. In response, the monitor displays a message alerting the patient to stop moving or a game in which the objective requires the patient to hold his or her finger still. The device can delay measurement until the patient is still enough and remains still long enough for a clear measurement.

### PRIORITY CLAIM TO RELATED PROVISIONAL APPLICATIONS

The present application claims priority benefit under 35 U.S.C. §119(e) to U.S. Provisional Patent Application Ser. No. 61/476,694 filed Apr. 18, 2011, titled Pediatric Monitor Sensor Steady Game, the disclosure of which are hereby incorporated in their entirety by reference herein.

### FIELD OF THE DISCLOSURE

The disclosure herein relates generally to patient monitoring with non-invasive spot-check medical devices. More specifically, the present disclosure relates to devices and methods for aiding a patient in remaining still during monitoring for more accurate results.

### BACKGROUND

The standard of care in caregiver environments includes patient monitoring through spectroscopic analysis using, for example, a pulse oximeter. Devices capable of spectroscopic analysis generally include a light source(s) transmitting optical radiation into or reflecting off a measurement site, such as, body tissue carrying pulsing blood. After attenuation by tissue and fluids of the measurement site, a photodetection device(s) detects the attenuated light and outputs a detector signal(s) responsive to the detected attenuated light. A signal processing device(s) process the detector(s) signal(s) and outputs a measurement indicative of a blood constituent of interest, such as glucose, oxygen, met hemoglobin, total hemoglobin, other physiological parameters, or other data or combinations of data useful in determining a state or trend of wellness of a patient.

In noninvasive devices and methods, a sensor is often adapted to position a finger proximate the light source and light detector. For example, noninvasive sensors often include a clothespin-shaped housing that includes a contoured bed conforming generally to the shape of a finger. The contoured bed positions the finger for measurement and attempts to stabilize it.

A drawback to such devices is that, sometimes, for accurate readings, the patient must remain substantially still and must keep his or her finger and the sensor at a certain angle. Patient motion or movement can cause inaccurate monitoring or signal dropout.

### SUMMARY

This disclosure describes embodiments of noninvasive methods, devices, and systems for measuring physiologically relevant patient characteristics and for helping the patient remain still during monitoring. When the device detects that the patient is moving or not in the appropriate position, it can delay measurement until it determines there is a window of time for clear and accurate measurements. In certain embodiments, when the device detects movement or inappropriate positioning, a display on a monitor alerts the patient to stop moving and/or reposition his or her finger. In other embodiments, the display on the monitor displays a game for the patient to play. The patient can accomplish the game objective by holding his or her finger still in the appropriate position.

For purposes of summarizing the disclosure, certain aspects, advantages and novel features have been described herein. It is to be understood that not necessarily all such advantages can be achieved in accordance with any particular embodiment disclosed herein. Thus, the disclosure described herein can be embodied or carried out in a manner that achieves or optimizes one advantage or a group of advantages as taught herein without necessarily achieving other advantages as can be taught or suggested herein.

### BRIEF DESCRIPTION OF THE DRAWINGS

Throughout the drawings, reference numbers can be re-used to indicate correspondence between referenced elements. The drawings are provided to illustrate embodiments described herein and not to limit the scope thereof

FIG. 1 illustrates an exemplary handheld monitor and an exemplary noninvasive optical sensor of a patient monitoring system, according to embodiments of the disclosure;

FIG. 2 shows a monitoring device in accordance with one embodiment with the sensor attached to a patient's finger;

FIG. 3 illustrates an exemplary embodiment of a movement or balance game involving a teddy bear on a tight rope according to embodiments of the disclosure;

FIG. 4 illustrates an exemplary embodiment of a movement or balance game involving a sun in a sky according to embodiments of the disclosure;

FIG. 5 illustrates an exemplary embodiment of a movement or balance game involving a car or cars on a road or racetrack according to embodiments of the disclosure;

FIG. 6 illustrates an exemplary embodiment of a movement or balance game involving balloon floating in the air according to embodiments of the disclosure;

FIG. 7 illustrates an exemplary embodiment of a movement or balance game involving a person balancing an object on his head according to embodiments of the disclosure.

### DETAILED DESCRIPTION

A monitoring device in accordance with one embodiment of the present disclosure comprises a physiological sensor including a detector housing for attachment to a measurement site. The measurement site can be, for example, a patient's finger, and the detector housing can comprise a clothespin-shaped housing that includes a contoured bed conforming generally to the shape of a finger. The monitoring device further comprises one or more monitors that process and/or display the sensor's output and/or other information. The monitors can include various components, such as a sensor front end, a signal processor, a display, etc.

The sensor can be integrated with a monitor, for example, into a handheld unit including the sensor, a display and user controls. In other embodiments, the physiological sensor can be connected to and communicate with the monitor(s) via a cable or a wireless connection. The communication can be via wire(s), cable(s), flex circuit(s), wireless technologies, or other suitable analog or digital communication methodologies and devices to perform those methodologies. Many arrangements allow the sensor to be attached to the measurement site while the monitor is attached elsewhere on a patient, such as the patient's arm, placed at a location near the patient, such as a bed, shelf or table, or held by the patient.

The physiological sensor further comprises a 3D accelerometer that detects motion of the physiological sensor. The 3D accelerometer can also be connected to and communicate with the monitor(s) via the cable or wireless connection. The monitor(s) can also process and/or display information related to the 3D accelerometer's output. The sensor, 3D accelerometer, and/or or monitor(s) can also provide outputs to a storage device or network interface.

Reference will now be made to the Figures to discuss embodiments of the present disclosure.

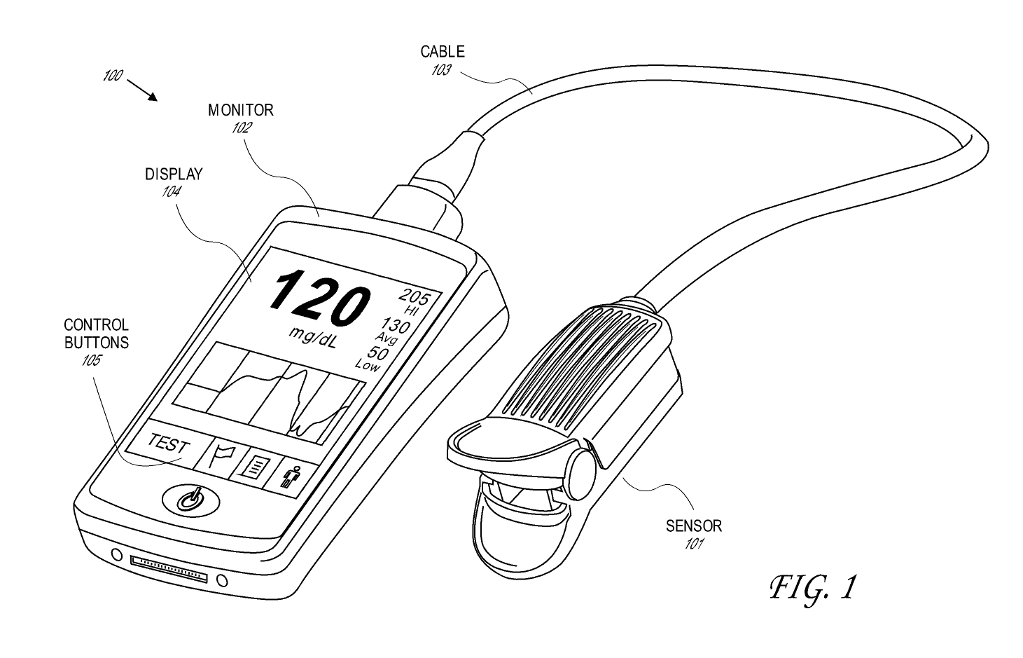

FIG. 1 illustrates one example of a monitoring device 100. In the depicted embodiment, the monitoring device 100 includes a finger clip sensor 101 connected to a monitor 102 via a cable 103. The finger clip sensor 101 includes a 3D accelerometer. In the embodiment shown, the monitor 102 includes a display 104, control buttons 105, and a power button. Moreover, the monitor 102 can advantageously include electronic processing, signal processing, and data storage devices capable of receiving signal data from said sensor 101, processing the signal data to determine one or more output measurement values indicative of one or more physiological parameters of a monitored patient, and displaying the measurement values, trends of the measurement values, combinations of measurement values, and the like. The electronic processing, signal processing, and data storage devices of the monitor 102 can also be capable of receiving data from the 3D accelerometer, processing the data, generating displays based at least in part on the data as further described below, and directing the system when to record measurements of the physiological parameters of interest.

The cable 103 connecting the sensor 101 and the monitor 102 can be implemented using one or more wires, optical fiber, flex circuits, or the like. In some embodiments, the cable 103 can employ twisted pairs of conductors in order to minimize or reduce cross-talk of data transmitted from the sensor 101 to the monitor 102. Various lengths of the cable 103 can be employed to allow for separation between the sensor 101 and the monitor 102. The cable 103 can be fitted with a connector (male or female) on either end of the cable 103 so that the sensor 101 and the monitor 102 can be connected and disconnected from each other. Alternatively, the sensor 101 and the monitor 102 can be coupled together via a wireless communication link, such as an infrared link, radio frequency channel, or any other wireless communication protocol and channel.

The monitor 102 can be attached to the patient. For example, the monitor 102 can include a belt clip or straps that facilitate attachment to a patient's belt, arm, leg, or the like. The monitor 102 can also include a fitting, slot, magnet, LEMO snap-click connector, or other connecting mechanism to allow the cable 103 and sensor 101 to be attached to the monitor 102.

The monitor 102 can also include other components, such as a speaker, power button, removable storage or memory (e.g., a flash card slot), an AC power port, and one or more network interfaces, such as a universal serial bus interface or an Ethernet port. For example, the monitor 102 can include a display 104 that can indicate measurements obtained by the physiological sensor, prompt a user for input, or display messages and/or games as discussed below.

In addition, although a single sensor 101 with a single monitor 102 is shown, different combinations of sensors and device pairings can be implemented. For example, multiple sensors can be provided for a plurality of differing patient types or measurement sites or even patient fingers.

Example monitoring devices are described in U.S. Publication No. 2010/0030040, filed Aug. 3, 2009, titled “Multi-Stream Data Collection System for Noninvasive Measurement of Blood Constituents” and U.S. Publication No. 2010/0010326, filed Jul. 2, 2009, titled “Contoured Protrusion for Improving Spectroscopic Measurement of Blood Constituents,” the disclosures of which are both hereby incorporated by reference in their entirety. An example commercial embodiment of a monitoring device is available from Masimo® Corporation of Irvine, Calif. under the trademark Pronto-7™.

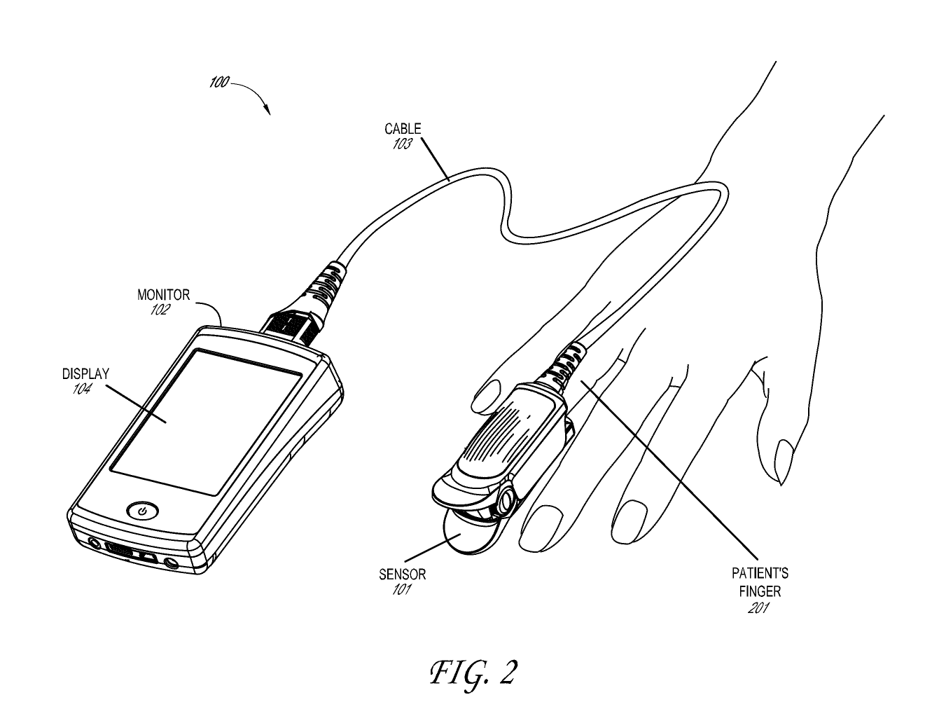

FIG. 2 shows a monitoring device 100 in accordance with one embodiment of the present disclosure with the sensor attached to a patient's finger 201.

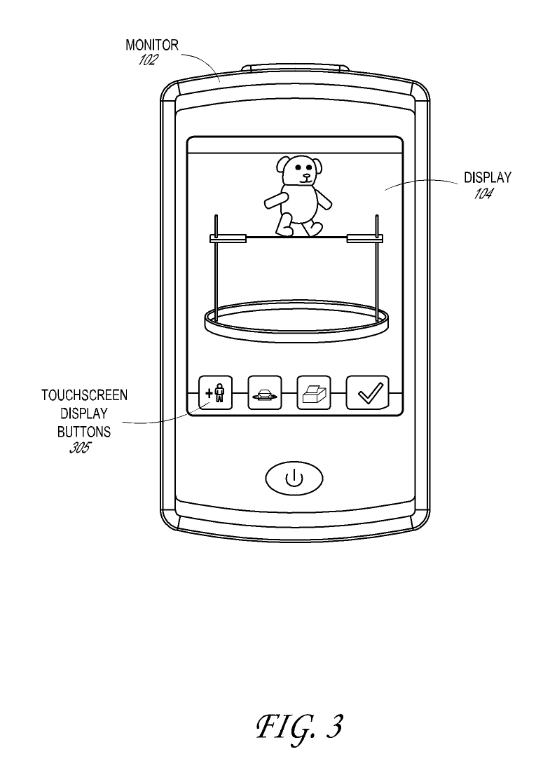

In operation, when the 3D accelerometer detects that the patient and physiological sensor are moving and/or not in the appropriate position for accurate measurement, the system delays measurement and triggers an appropriate display on the monitor's 102 display 104. Before attaching the physiological sensor to the patient and beginning monitoring, a user can input information regarding the patient, e.g., whether the patient is an adult or child, into the monitor 102 via, for example, control buttons 105 on the monitor housing or touch screen display buttons 305 (shown in FIG. 3). Alternatively, a user can select whether the system is to display a message or game as described below. In some embodiments, the system displays a message on the display 104 prompting the user to input such information regarding the patient or type of display desired.

For an adult or adolescent patient, when the 3D accelerometer detects that the patient is moving or that the sensor is not in the appropriate position, the display 104 can display a message alerting the patient to stop moving or adjust his or her finger. The system can also delay measurement until there is an appropriate window of time to obtain a clear measurement. In some embodiments, the 3D accelerometer detects the direction or manner in which the patient is moving and/or how the finger is positioned incorrectly. The display 104 can provide information explaining how the patient is moving, for example, tapping the finger up and down, moving the finger from side to side, shaking or vibrating, etc. The display 104 can also provide information directing the patient how to adjust his or her finger for more accurate measurement, for example, directing the patient to tilt the finger up or down.

This feature provides important benefits as a patient may not be consciously aware that he or she is moving in a certain way. Providing specific information regarding the type of motion can help the patient correct for it and allow the system to proceed to monitoring more quickly and easily. Once the patient is still and positioned appropriately, the display can display a message directing the patient to remain still in that position. The system can then begin monitoring the physiological parameter(s) of interest.

In alternative embodiments for pediatric patients, the display 104 displays and initiates a movement or balance game rather than a verbal message in response to the detection of motion or inappropriate positioning. A young child may not be able to read a message directing him or her to stop moving. Even if the child could read such a message, children often have difficulty remaining still even if asked or directed to do so. Therefore, the display 104 is configured to display one or more games such that the patient is motivated by the game to keep his or her finger still in the appropriate position. In addition to helping the patient remain still so that the monitoring device can obtain accurate readings, such games advantageously provide an activity to occupy and possibly distract the patient.

In various embodiments, the game can be designed such that the patient accomplishes the objective of the game by holding his or her finger still and at the appropriate angle for accurate measurement. In some embodiments, the patient can earn points in the game for remaining still in the appropriate position.

For example, in one embodiment illustrated in FIG. 3, the game display comprises images of a teddy bear on a tightrope. Movement of the patient's finger and therefore the sensor corresponds to and causes movement of the bear with respect to the tightrope. The patient keeps his or her finger substantially still and in the proper position to keep the bear properly balanced on the tightrope. In some embodiments the bear remains in the same position on the tightrope. In other embodiments the bear travels from one side of the tightrope to the other, for example on foot, on a bicycle, or in some other way. The patient remains substantially still for the bear to maintain balance and reach the opposite side. The timing of the bear's travel across the tight rope can correspond to the time the monitor requires to make an accurate measurement. If the patient moves such that the measurement must be restarted, the game can illustrate the bear falling off the rope and starting over at one side of the rope. In other similar embodiments, the bear can be replaced by an animal or human and the tightrope can be replaced by a balance beam, among other things. In an embodiment, the patient receives points for how quickly he brings the bear to a balanced position and the extent to which he is able to maintain that balanced position. In an embodiment, the patient can receive bonus points for getting the bear to travel from one side of the rope, balance beam, etc., to the other side without falling on the first try. Of course, other variations of the game will be apparent based on the disclosure herein.

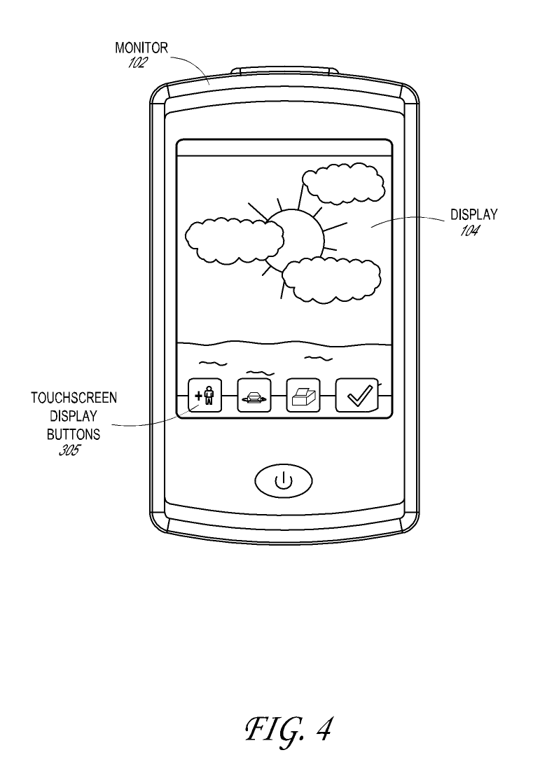

In another embodiment illustrated in FIG. 4, the game display comprises images of a sun in a sky. Movement of the patient's finger and sensor causes clouds to appear and thicken, covering the sun and dimming its light. Remaining still in the proper position causes the clouds to lessen and eventually disappear, allowing the sun to shine more brightly. The objective for the patient is for the sky to be clear and the sun to shine as brightly as possible. In an embodiment, the patient can receive points in the game corresponding to the brightness of the sun and the length of time the brightest state is maintained.

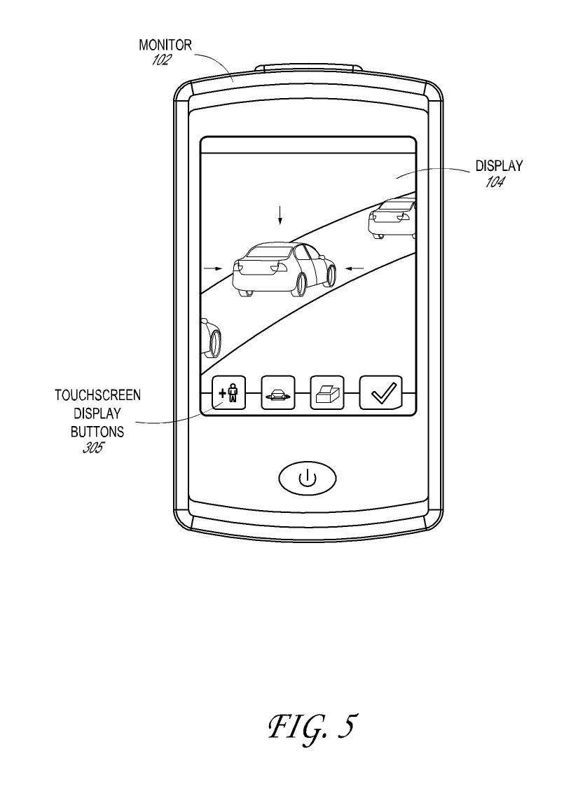

FIG. 5 illustrates yet another embodiment. Here, the game display comprises a car traveling on a road or racetrack. The display can also include other cars surrounding the car whose movement corresponds to movement of the patient's finger and sensor. The objective is to keep the car on the road or racetrack and/or prevent it from hitting or being hit by other cars. In an embodiment, moving the finger to one side or the other causes the car to veer off the road or racetrack, moving the finger up causes the car to speed up and hit a car in front of it, and moving the finger down causes the car to slow or stop such that it is hit by a car traveling behind it. The patient can receive points for keeping the car at a steady speed and avoiding veering off the road or collisions with other cars.

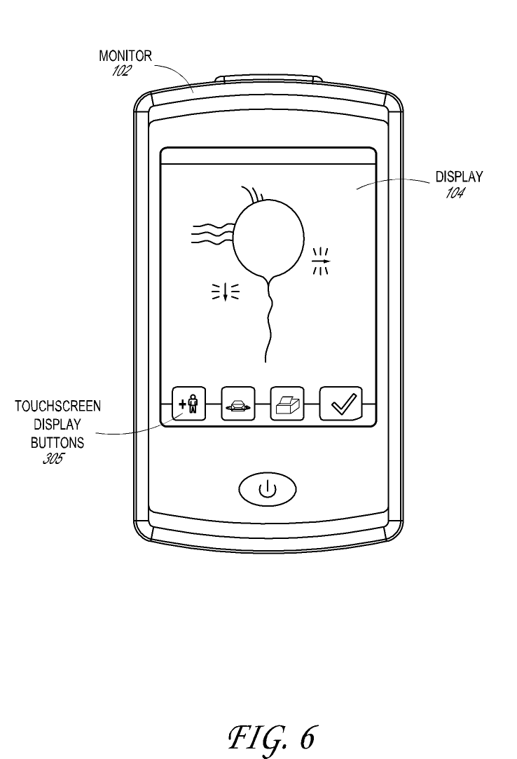

In another embodiment shown in FIG. 6, the game display comprises a balloon floating in the air. The objective is to keep it stationary, and movement of the patient's finger can cause the balloon to move, pop, and/or deflate. Alternatively, the game display can comprise a human or animal holding a balloon by a string. Movement of the patient's finger causes the balloon to be blown away, pop, or deflate. In an embodiment, the patient receives points for keeping the balloon inflated and not letting it float out of position. In an embodiment, a child holds a pin close to the balloon and movement of the finger can cause the balloon to get close to or touch the pin making it pop.

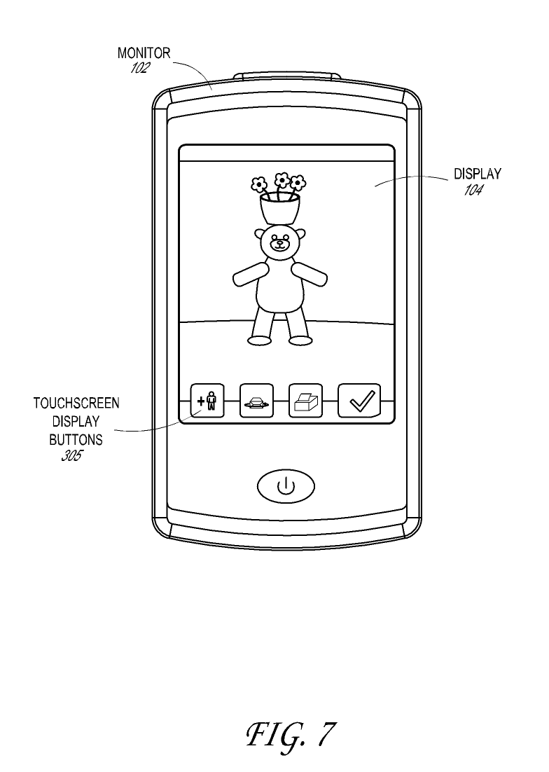

In other embodiments, such as the embodiment illustrated in FIG. 7, the game display can comprise an animal or person balancing an object on his head or in his hands. Movement of the patient's finger causes the object to become unbalanced such that the animal or person might drop the object. The patient can receive points corresponding to how well he keeps the object balanced.

Although the games described herein are primarily intended for use with pediatric patients, they can also be used for adult or adolescent patients as well. This is beneficial in a situation where, for example, an adult or adolescent patient is nervous or anxious and therefore having difficulty remaining still in response to a message or if the patient wants to play a game as a distraction. In some embodiments, the system can include games more appropriate for adult or adolescent patients. For example, a game might comprise a person meditating and the patient holds his or her finger still in the appropriate position to keep the person in a meditative state and/or earn points.

The games discussed above are example embodiments and not intended to be limiting. Any game wherein the patient can accomplish the objective by keeping his or her finger still in the appropriate position can be effective.

One or more of the games disclosed above or similar games can be stored in the monitor's 102 data storage device. Additional games can be loaded to the monitor via a removable storage or memory (e.g., a flash card) or a network interface.

After a user provides input to the monitor 102 indicating that the patient is a child or that a game is to be used rather than a verbal alert message, the display 104 can provide various game options available and prompt the user or patient to select one via a touch screen display or control buttons 105.

In operation, when the patient has remained still for a period of time sufficient for the sensor 101 to obtain a clear and complete measurement, the display 104 can display a message indicating that monitoring is complete or can display a final display of the game, for example indicating the number of points the patient earned. A user can then use a touch screen or control buttons 105 on the monitor 102 to change the display to, for example, the measured physiological parameters.

In some embodiments, a data storage device included in the monitor 102 can store, for example, the number of points the patient receives when playing a game. The next time the patient requires monitoring with the monitoring device 100, the patient can try to improve his game performance as compared to the previous monitoring session, for example, by achieving and maintaining an appropriate and sufficiently stationary state for accurate monitoring more quickly. Alternatively, the display can display the patient's points compared to other patients using the device or similar devices. In an embodiment, the patient can receive this information via an email or the like generated by the device or a server in communication with the device.

While a number of preferred embodiments and variations thereof have been described in detail, other modifications and methods of using and medical applications for the same will be apparent to those of skill in the art. Accordingly, it should be understood that various applications, modifications, and substitutions can be made of equivalents without departing from the spirit of the disclosure or the scope of the claims.

What is claimed is:

1. A noninvasive medical monitoring device, the monitoring device comprising:
a sensor having a housing comprising an upper shell and a lower shell pivotally connected together, the upper and lower shells each shaped to accept body tissue of a medical patient;
one or more emitters disposed in the housing, the one or more emitters configured to impinge light on the body tissue of the patient;
one or more detectors disposed in the housing, the one or more detectors configured to receive the light after attenuation by the body tissue of the patient and to output one or more intensity signals responsive to the attenuated light;
a 3D accelerometer disposed in the housing, the 3D accelerometer configured to detect movement of the sensor and to output data related to the detected movement; and
a monitor configured to receive output from said physiological sensor and said 3D accelerometer;
wherein the monitor comprises a display and content displayed on the display is based at least in part on output from said 3D accelerometer.

2. The noninvasive medical monitoring device of claim 1 wherein the sensor is connected to and communicates with the monitor via a cable.

3. The noninvasive medical monitoring device of claim 1 wherein the sensor is connected to and communicates with the monitor via a wireless connection.

4. The noninvasive medical monitoring device of claim 1 wherein content displayed on the display comprises one or more verbal messages.

5. The noninvasive medical monitoring device of claim 1 wherein content displayed on the display comprises a movement or balance game.

6. The noninvasive medical monitoring device of claim 1 wherein the monitor further comprises a data storage device.

7. The noninvasive medical monitoring device of claim 1 wherein the monitor further comprises a network interface.

8. A noninvasive medical monitoring device, the monitoring device comprising:
a physiological sensor;
a 3D accelerometer associated with the physiological sensor, the 3D accelerometer configured to detect movement of the sensor and to output data related to the detected movement; and
a monitor comprising a display and a processor;
wherein the processor is configured to process output data from the 3D accelerometer related to the detected movement and generate content based at least in part on said output from said 3D accelerometer.

9. The noninvasive medical monitoring device of claim 8 wherein the sensor and 3D accelerometer are connected to and communicate with the monitor via a cable.

10. The noninvasive medical monitoring device of claim 8 wherein the sensor and 3D accelerometer are connected to and communicate with the monitor via a wireless connection.

11. The noninvasive medical monitoring device of claim 8 wherein content generated by the processor comprises one or more verbal messages.

12. The noninvasive medical monitoring device of claim 8 wherein content generated by the processor comprises a movement or balance game.

13. The noninvasive medical monitoring device of claim 8 wherein the monitor further comprises a data storage device.

14. The noninvasive medical monitoring device of claim 8 wherein the monitor further comprises a network interface.

15. The noninvasive medical monitoring device of claim 8 wherein the processor is further configured to display a message on the display prompting a user for input regarding a type of content to be generated.

16. A method of measuring one or more physiological parameters of a patient, said method comprising:
detecting movement and position of a measurement site on a patient with a sensor, wherein said sensor is connected to and communicates with a monitor;
displaying content on a display on said monitor based at least in part on the detected movement and position of said measurement site;
emitting optical radiation to said measurement site on said patient;
detecting attenuated optical radiation from said measurement site on said patient; and
determining an output measurement value indicative of a physiological parameter based on the detected attenuated optical radiation.

17. The method of claim 16, further comprising delaying the emitting of said optical radiation to said measurement site on said patient until the detected movement and position of said measurement site are within acceptable limits.

18. The method of claim 16, further comprising prompting a user for input regarding a type of content to be displayed on said monitor.

19. The method of claim 16, further comprising storing data related to the detected movement and position of said measurement site over time.

20. The method of claim 16, further comprising outputting data related to the detected movement and position of said measurement site over time.
